(19) United States
(12) Patent Application Publication
(10) Pub. No.: US 2007/0122483 A1
(43) Pub. Date: May 31, 2007

(54) Fluocinolone acetonide drug substance polymorphic interconversion

(76) Inventor: Sharon Myers, Webster, NY (US)

Correspondence Address: Bausch & Lomb Incorporated, One Bausch & Lomb Place, Rochester, NY, 14604-2701, US

(21) Appl. No.: 11/604,552

(22) Filed: Nov. 27, 2006

Related U.S. Application Data
(60) Provisional application No. 60/740,337, filed on Nov. 29, 2005.

(51) Int. Cl. A61K 9/14

(52) U.S. Cl. 424/489

(57) ABSTRACT

Disclosed in embodiments herein is a method of providing the same form of FA. Regardless of which form or mixture of forms are present by forming a slurry from samples of fluocinolone acetonide containing mostly Form A and little to no Form A.

### PRIORITY CLAIMS

None

### CROSS-REFERENCE TO RELATED APPLICATIONS

None

### BACKGROUND AND SUMMARY

Fluocinolone acetonide, a powerful anti-inflammatory steroid, is practically insoluble in water. FA has been used in the formulation of anti-inflammatory topical products for more than 20 years. Bausch & Lomb Incorporated (B & L) and Control Delivery Systems (CDS) have selected Sicor—Societa Italiana Corticosteroidi S.p.A., Via Terrazzano, 77-20017 RHO (Milano), Italy, as the principal supplier for fluocinolone acetonide (FA), USP, EP.

Polymorphism is commonly defined as the ability of a substance to exist in two or more crystalline phases that differ in the arrangement and/or conformation of molecules in the crystal lattice. A classic example is pure carbon which can exist as graphite or diamond. Materials having the same chemical composition but dramatically differing properties. The majorities of the pharmaceutical products on the market exist in the solid form and therefore demonstrate some degree of polymorphism.

Physical form of pharmaceutical active agents is important for a variety of reasons. Solid state properties include: physical properties (color, dissolution); chemical properties (reactivity, stability); regulatory issues (safety, efficacy); product performance (consistency); as well as intellectual property (new products, patents).

In 1997, Bartolomei, et al. reported that FA appeared to exist in three different crystalline forms (referred to as A, B and C) with unique physical, thermal, and spectroscopic behavior. Bartolomei, et al. report that form B can be obtained by crystallization from hot ethanol on a water bath. The same crystallization was also used to obtain form A by crystallization from cold acetone, cold chloroform and cold methanol. Form B can be prepared by crystallization from solvents such as cold absolute ethanol, whether or not water was present. Crystallization from ethyl acetate or 2:1 acetone/hexane mixtures or by freeze drying the FA solution at room temperature resulted in formation of form C. Upon heating any of the three forms to above 210 degrees C. form B was obtained.

Thus it would be an improvement to the art to be able to provide drug compositions in predominately a single form without having to resort to use of organic solvents.

Disclosed in embodiments herein is a method of providing the same form of FA. Regardless of which form or mixture of forms are present by forming a slurry from samples of fluocinolone acetonide containing mostly Form A and little to no Form A.

### BRIEF DESCRIPTION OF THE DRAWINGS

FIG. 1 is a solid-state NMR 13C CP/MAS NMR spectrograph of the primary form, mixture and secondary form of FA;

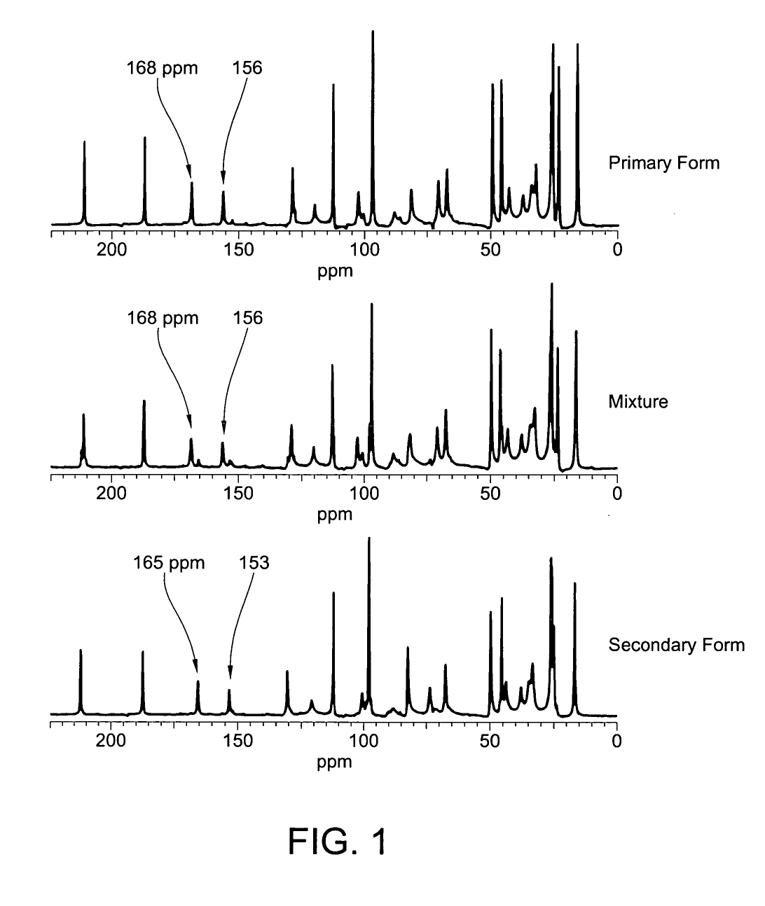

FIG. 2 is a solid-state NMR 13C CP/MAS NMR spectrograph of the mixture slurry and secondary form slurry after treatment according to the invention herein.

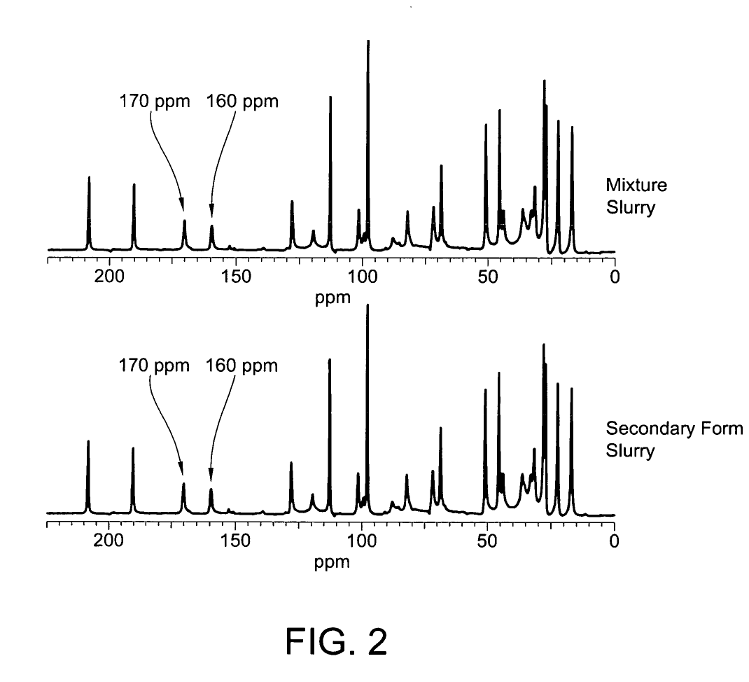

### DETAILED DESCRIPTION

Disclosed in embodiments herein is a method of providing the same form of FA. Regardless of which form or mixture of forms are present by forming a slurry from samples of fluocinolone acetonide containing mostly Form A and little to no Form A.

Slurries containing drug substance in water were prepared and covered and stirred for a sufficient length of time to allow the formation of a hydrate to occur. The ratio of drug substance to water can vary depending upon the particular drug substance. Suitable ranges of drug to water in the slurry can vary between 1 and 99 percent by weight, more preferably 1 to 50 percent by weight, even more preferably between 1 and 10 percent by weight.

The water used for forming the slurry may be any water approved for use with pharmaceutical products. For example, sterile water for injection.

The slurry is allowed to mix for a time sufficient to allow the formation of a hydrate to occur. Depending upon the active, the slurry mixing time can be up to about 100 days, more preferably up to about 50 days, even more preferably up to about 21 days. The amount of time necessary for the slurry to result in formation of the desired hydrate will readily determined by one of ordinary skill in the art without undue experimentation.

To remove the water from the slurry any suitable method such as is known to those of ordinary skill in the art may be used. For example, the samples may be filtered and dried overnight at room temperature, dried under desiccant conditions or any other suitable method.

Characterization of the hydrate can be by any suitable method including, but not limited to, Chromatography, HPLC, GC, TLC; Optical Microscopy; Mass Spectrometry, MS, hyphenated methods; Magnetic Resonance Spectroscopy, NMR; X-ray Diffraction, XRPD; Thermal Methods, TGA, DSC; and FTIR, Raman.

Solid-state NMR is a preferred method to characterize pharmaceutical solids. Its advantages include: 1.Nondestructive and noninvasive; 2.No calibration standards; 3. Particle size is not an issue; 4. Mixture analysis of solid forms and 5. Quantification of forms. In addition, regulatory authorities now recognize the importance of solid-state NMR spectroscopy and how the technique is linked to the drug development process.

The invention will now be explained by the following example which is intended to illustrate but not limit the invention in any way.

### EXAMPLES

### Example 1

Sample Preparation

Slurries containing 0.5 g FA in 10 ml of water were covered and stirred at room temperature for seven days. To remove the water, the samples were filtered and dried overnight at room temperature.

Experimental Conditions

Approximately 80-100 mg of sample was packed inside a 7 mm rotor. Magic-angle spinning (MAS) NMR spectra of fluocinolone acetonide (FA) were acquired using a Varian Unity Inova 400 operating at 399.8 and 100.5 MHz for ¹H and ¹³C respectively. The spectra were acquired using cross-polarization (CP) with total sideband suppression and a spinning speed of 4 kHz. Additional acquisition and processing parameters are noted below:

| Parameter/Hardware | ¹³C NMR | |
|---|---|---|
| Probe | Varian CP/MAS | |
| Spectral Width | 35.3 | kHz |
| Number of Data Points | 2048 | |
| Transmitter Power | 63 | dB |
| Pulse Width | 4.5 | us |
| Recycle Delay | 10 | sec |
| Contact Time | 2 | ms |
| Number of Acquisitions | 5000 | |
| Line Broadening | 20 | Hz |
| Fourier Number | 8192 | |

| | |
|---|---|
| Filter | Digital w/oversampling |

All spectra were externally referenced to hexamethylbenzene (HMB) (¹³C, aliphatic peak at 17.4 ppm).

### CONCLUSION

In this work, 13C CP/MAS NMR was used to investigate the polymorphic changes that occur in FA after exposure to water for an extended time period. The results indicate that under hydration all polymorphic forms interconvert to the same form.

The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

What is claimed is:

1. A method for providing a single state of a solid pharmaceutically active substance, the method comprising:
providing a pharmaceutically active substance in a powdered form;
providing an amount of water suitable for use in pharmaceutical processing;
combining the pharmaceutically active substance and the water to provide a slurry;
mixing the slurry for a sufficient time for a single state of the pharmaceutically active substance to form; and,
removing the water from the slurry.

2. The method of claim 1 further comprising the step of characterizing the pharmaceutically active substance after removal of the water to determine if the substance is present in a single form.

3. The method of claim 1 wherein the slurry contains up to about 99 percent by weight of the pharmaceutically active substance.

4. The method of claim 1 wherein the slurry contains up to about 50 percent by weight of the pharmaceutically active substance.

5. The method of claim 1 wherein the slurry contains up to about 10 percent by weight of the pharmaceutically active substance.

6. The method of claim 1 wherein the slurry is mixed for up to about 100 days.

7. The method of claim 1 wherein the slurry is mixed for up to about 50 days.

8. The method of claim 1 wherein the slurry is mixed for up to about 21 days.

9. The method of claim 1 wherein the water is removed by filtering and drying overnight at room temperature

10. The method of claim 1 wherein the water is removed by desiccant conditions.

11. The method of claim 2 wherein the sample is characterized by a method selected from the group consisting of Chromatography, HPLC, GC, TLC; Optical Microscopy; Mass Spectrometry, MS, hyphenated methods; Magnetic Resonance Spectroscopy, NMR; X-ray Diffraction, XRPD; Thermal Methods, TGA, DSC; and FTIR, Raman.
